(19) United States
(12) Patent Application Publication
(10) Pub. No.: US 2022/0365228 A1
(43) Pub. Date: Nov. 17, 2022

(54) RADIATION IMAGING APPARATUS, RADIATION IMAGING SYSTEM, DRIVE METHOD FOR RADIATION IMAGING APPARATUS, AND NON-TRANSITORY COMPUTER-READABLE STORAGE MEDIUM

(71) Applicant: CANON KABUSHIKI KAISHA, Tokyo (JP)

(72) Inventors: Kanako Sato, Kanagawa (JP); Akira Tsukuda, Tokyo (JP)

(21) Appl. No.: 17/815,298

(22) Filed: Jul. 27, 2022

Related U.S. Application Data
(63) Continuation of application No. PCT/JP2021/006161, filed on Feb. 18, 2021.

(30) Foreign Application Priority Data
Feb. 26, 2020 (JP) 2020-030848

(51) Int. Cl. G01T 1/17; H04N 5/378; H04N 5/3745; G01T 1/20

(52) U.S. Cl. CPC G01T 1/17; H04N 5/378; H04N 5/3745; G01T 1/20184

(57) ABSTRACT

A radiation imager comprising pixels each including a converter to generate a signal, a sampling circuit and a processor is provided. The sampling circuit samples the signal with first sensitivity and with second sensitivity higher than the first sensitivity. If a first signal value obtained by sampling the signal with the first sensitivity is smaller than a first threshold, the processor generates a pixel value based on a second signal value obtained by sampling the signal with the second sensitivity, if the first signal value exceeds a second threshold larger than the first threshold value, the processor generates a pixel value based on the first signal value, and if the first signal value is not less than the first threshold and not more than the second threshold, the processor generates a pixel value based on the first and second signal values.

### CROSS-REFERENCE TO RELATED APPLICATIONS

This application is a Continuation of International Patent Application No. PCT/JP2021/006161, filed Feb. 18, 2021, which claims the benefit of Japanese Patent Application No. 2020-030848, filed Feb. 26, 2020, both of which are hereby incorporated by reference herein in their entirety.

### BACKGROUND OF THE INVENTION

### Field of the Invention

The present invention relates to a radiation imaging apparatus, a radiation imaging system, a drive method for the radiation imaging apparatus, and a non-transitory computer-readable storage medium.

### Background Art

A radiation imaging apparatus including a flat panel type pixel panel on which pixels, each having a combination of a conversion element and a switch element such as a thin-film transistor (TFT), are arrayed has been widely used. PTL 1 describes that a capacitance for sensitivity switching is connected to an output node of a photodiode via a switch, and a high dynamic range mode and a high sensitivity mode are switched by switching ON/OFF of the switch.

### CITATION LIST

### Patent Literature

PTL 1: Japanese Patent Laid Open No. 2002-344809

In a radiation image, a region with a high dose and a region with a low dose can be generated in accordance with an object. In a low dose region where the dose is low, the linearity of an A/D-converted signal value with respect to the incident dose can deteriorate due to an influence of 1/f noise generated in a pixel or an A/D converter, or the like. In a high dynamic range mode in which a capacitance for sensitivity switching is connected to an output node of a photodiode to increase the dynamic range, the signal value in the low dose region becomes smaller than in a high sensitivity mode. Accordingly, the influence of 1/f noise or the like relatively increases.

The present invention has as its object to provide a technique advantageous in, in a radiation imaging apparatus, suppressing deterioration of the linearity in a low dose region in high dynamic range.

### SUMMARY OF THE INVENTION

According to some embodiments, a radiation imaging apparatus that comprises a plurality of pixels each including a converter configured to generate a pixel signal corresponding to incident radiation, a sampling circuit configured to sample the pixel signal, and a processor configured to process a signal sampled by the sampling circuit, wherein for each pixel of the plurality of pixels, the sampling circuit is configured to sample the pixel signal with first sensitivity and with second sensitivity higher than the first sensitivity, and if a first signal value obtained by sampling the pixel signal with the first sensitivity by the sampling circuit is smaller than a first threshold value, the processor is configured to generate a pixel value based on a second signal value obtained by sampling the pixel signal with the second sensitivity by the sampling circuit, if the first signal value exceeds a second threshold value larger than the first threshold value, the processor is configured to generate a pixel value based on the first signal value, and if the first signal value is not less than the first threshold value and not more than the second threshold value, the processor is configured to generate a pixel value based on the first signal value and the second signal value, is provided.

According to some other embodiments, a drive method for a radiation imaging apparatus that comprises a plurality of pixels each including a converter configured to generate a pixel signal corresponding to incident radiation, the method including sampling the pixel signal with first sensitivity and second sensitivity higher than the first sensitivity, and processing a signal sampled in the sampling the pixel, wherein in the processing the signal, for each pixel of the plurality of pixels, if a first signal value obtained by sampling the pixel signal with the first sensitivity is smaller than a first threshold value, a pixel value is generated based on a second signal value obtained by sampling the pixel signal with the second sensitivity, if the first signal value exceeds a second threshold value larger than the first threshold value, a pixel value is generated based on the first signal value, and if the first signal value is not less than the first threshold value and not more than the second threshold value, a pixel value is generated based on the first signal value and the second signal value, is provided.

According to still other embodiments, a non-transitory computer-readable storage medium storing a program for causing a computer to execute a drive method for a radiation imaging apparatus that comprises a plurality of pixels each including a converter configured to generate a pixel signal corresponding to incident radiation, the method including sampling the pixel signal with first sensitivity and second sensitivity higher than the first sensitivity, and processing a signal sampled in the sampling the pixel signal, wherein in the processing the signal, for each pixel of the plurality of pixels, if a first signal value obtained by sampling the pixel signal with the first sensitivity is smaller than a first threshold value, a pixel value is generated based on a second signal value obtained by sampling the pixel signal with the second sensitivity, if the first signal value exceeds a second threshold value larger than the first threshold value, a pixel value is generated based on the first signal value, and if the first signal value is not less than the first threshold value and not more than the second threshold value, a pixel value is generated based on the first signal value and the second signal value, is provided.

Further features of the present invention will become apparent from the following description of exemplary embodiments with reference to the attached drawings.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is view showing a configuration example of a radiation imaging system including a radiation imaging apparatus according to an embodiment.

FIG. 2 is a view showing an arrangement example of a pixel of the radiation imaging apparatus shown in FIG. 1.

FIG. 3 is a timing chart for explaining a control example of drive of the radiation imaging apparatus including the pixel shown in FIG. 2.

FIG. 4 is a view showing an arrangement example of a sensor unit of the radiation imaging apparatus shown in FIG. 1.

FIG. 5 is a view showing an arrangement example of a readout circuit of the radiation imaging apparatus shown in FIG. 1.

FIG. 6 is a flowchart illustrating a method of generating a pixel value of the radiation imaging apparatus shown in FIG. 1.

FIG. 7 is a view showing a modification of the pixel shown in FIG. 2.

FIG. 8 is a timing chart for explaining a control example of drive of the radiation imaging apparatus including the pixel shown in FIG. 7.

FIG. 9 is a view showing a configuration example of a radiation imaging system including a radiation imaging apparatus according to an embodiment.

FIG. 10 is a view showing an arrangement example of a pixel and a readout circuit of the radiation imaging apparatus shown in FIG. 9.

FIG. 11 is a flowchart showing a modification of the flowchart of FIG. 6.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

Radiation according to the present invention can include not only α-rays, β-rays, and γ-rays that are beams generated by particles (including photons) emitted by radioactive decay but also beams having energy equal to or higher than the energy of these beams, for example, X-rays, particle beams, and cosmic rays.

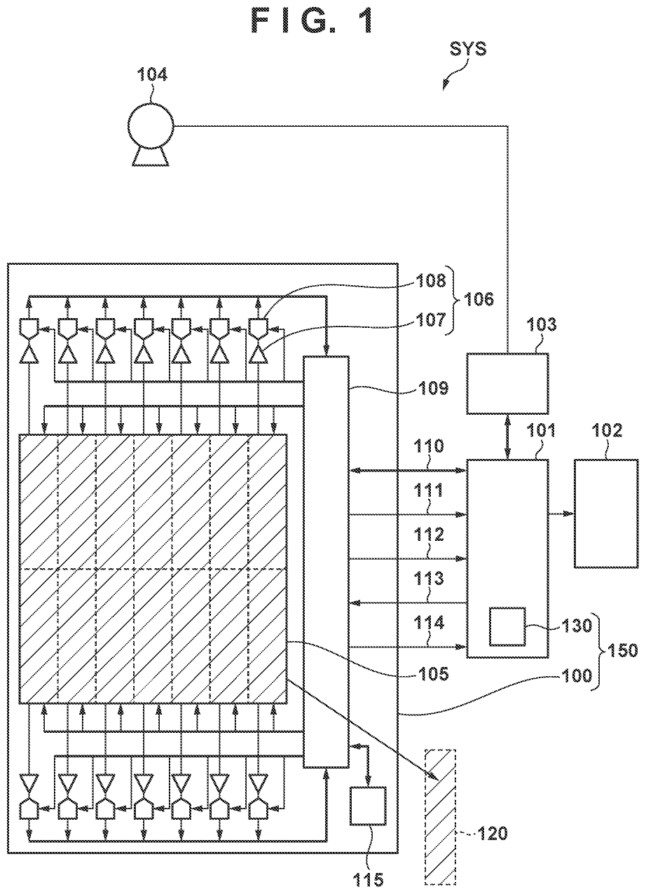

The arrangement of a radiation imaging apparatus and a drive method according to an embodiment will be described with reference to FIGS. 1 to 8. FIG. 1 is a system block diagram showing an example of the overall configuration of a radiation imaging system SYS including a radiation imaging apparatus 150 according to this embodiment. The radiation imaging system SYS includes an imaging unit 100, a system controller 101, a display 102, an irradiation controller 103, and a radiation source 104.

The imaging unit 100 acquires image data representing the internal information of an object by imaging using radiation, and outputs the image data to the system controller 101. The system controller 101 includes a processor 130 that performs image processing and data processing for the image data output from the imaging unit 100. Here, the radiation imaging apparatus 150 in this embodiment includes the imaging unit 100 for acquiring image data, and the processor 130 that performs image processing and data processing for the image data.

The system controller 101 also has a function as a controller that exchanges control signals among the respective units and performs system control and synchronization control for the overall radiation imaging system SYS including the imaging unit 100 and the irradiation controller 103. The display 102 includes, for example, a display, and displays radiation images based on the image data output from the imaging unit 100 via the system controller 101. For example, frame image data corresponding to irradiation with radiation is transferred from the imaging unit 100 to the system controller 101. The system controller 101 performs image processing for the frame image data. The display 102 then displays a radiation image in real time.

The system controller 101 controls the irradiation controller 103 so as to synchronize it with the imaging unit 100 at the time of capturing a radiation image. The irradiation controller 103 outputs a signal for irradiation with radiation to the radiation source 104 as a radiation generator in accordance with the control signal output from the system controller 101. The radiation source 104 generates radiation for radiation imaging in accordance with the signal output from the irradiation controller 103. In other words, the system controller 101 outputs, via the irradiation controller 103, a signal for controlling irradiation with radiation to the radiation source 104 for irradiating the imaging unit 100 of the radiation imaging apparatus 150 with radiation.

The imaging unit 100 includes a sensor panel 105, readout circuits 106, and a controller 109. The readout circuits 106 read out the image signals output from the sensor panel 105. The controller 109 controls each unit in the imaging unit 100 while exchanging signals such as control signals with the system controller 101.

A plurality of sensor units 120 are arrayed on the sensor panel 105. For example, each sensor unit 120 can be a sensor chip that is manufactured by a known semiconductor manufacturing process using a semiconductor substrate such as a silicon wafer and has a two-dimensional array of pixels that are CMOS type image sensors. Each sensor unit 120 has an imaging region for acquiring an image signal representing internal information of an object. In addition, each sensor unit 120 may have a light-shielded optical black region in addition to the imaging region. Each sensor unit 120 can be the one that is physically separated by dicing or the like. In other words, the plurality of sensor units 120 arrayed on the sensor panel 105 each can have a separable arrangement. Tiling the plurality of sensor units 120 on a plate-like base (not shown) can increase the size of the sensor panel 105. The respective sensor units 120 can be tiled such that the conversion elements of the pixels formed in the sensor units 120 are arrayed on both sides of the boundaries between the adjacent sensor units 120 at the same pitch as that of the interior portions of the sensor units 120. For the sake of descriptive convenience, in the arrangement shown in FIG. 1, the sensor units 120 are tiled in 2 rows×7 columns. However, the arrangement of the sensor panel 105 is not limited to this arrangement.

For example, a scintillator (not shown) for converting radiation into light is arranged on the incident surface side of the sensor panel 105 which is irradiated with radiation, and electrical signals corresponding to light converted from radiation are obtained by the conversion elements of the pixels arrayed on the respective sensor units 120 of the sensor panel 105. Although this embodiment will exemplify the arrangement of an imaging apparatus using pixels including indirect type conversion elements for converting radiation into light via a scintillator and photoelectrically converting the converted light, it is possible to use an imaging apparatus using direct type conversion elements for directly converting radiation into electrical signals.

Each readout circuit 106 includes, for example, a differential amplifier 107 and an A/D converter 108 for performing analog/digital (A/D) conversion. The arrangements and operations of the differential amplifiers 107 and the A/D converters 108 will be described later.

The upper side portion and lower side portion of the sensor panel 105 are provided with electrodes for exchanging signals or supplying power. The electrodes are connected to external circuits via a flying lead type printed wiring board (not shown). For example, the readout circuits 106 read out image signals from the sensor panel 105 via the electrodes. Control signals from the controller 109 are supplied to the sensor panel 105 via the electrodes.

The controller 109 controls the operations of the sensor panel 105, the differential amplifiers 107, and the A/D converters 108 to perform, for example, settings for a reference voltage supplied to each sensor unit 120, drive control of each pixel, and operation mode control. In addition, the controller 109 generates one frame data for each unit period by using image signals (digital data) A/D-converted by the A/D converters 108 of the readout circuits and output from the respective sensors of the sensor panel 105. The generated frame data is output as image data to the system controller 101.

The imaging unit 100 may further include a memory 115. The memory 115 may store a program for operating the imaging unit 100 of the radiation imaging apparatus 150, and the like. The memory 115 may also store various kinds of correction data and parameters.

The controller 109 and the system controller 101 exchange control signals such as control commands and image data via various types of interfaces. A control interface 110 is an interface for the exchange of imaging information and setting information such as drive modes and various types of parameters. The control interface 110 may exchange apparatus information such as the operation state of the imaging unit 100. An image data interface 111 is an interface for outputting an image signal (image data) output from the imaging unit 100 to the system controller 101. The controller 109 notifies the system controller 101 that the imaging unit 100 is set in the imaging enabled state by using a READY signal 112. The system controller 101 notifies the controller 109 of the timing of the start of irradiation with radiation (exposure) by using a synchronization signal 113 in accordance with the READY signal 112 output from the controller 109. The system controller 101 outputs a control signal to the irradiation controller 103 to start irradiation with radiation while an exposure permission signal 114 output from the controller 109 is enabled.

The above arrangement is configured to perform control of each unit, for example, drive control, synchronization control, and drive mode control in the radiation imaging system SYS. For example, an input unit (not shown) such as an information input unit or information input terminal that allows the user to input imaging information such as operation modes and various types of parameters may be connected to the system controller 101. Each unit is controlled based on imaging information input by the user. For example, the system controller 101 functions as a drive mode setting unit to select a drive mode based on the imaging information input by the user, and controls the overall radiation imaging system SYS so as to cause the radiation imaging system SYS to operate. The imaging unit 100 then generates frame data for each unit period based on an image signal from each pixel read out from the sensor panel 105 and outputs the frame data as image data to the system controller 101. The processor 130 of the system controller 101 performs predetermined image processing and data processing for the image data, and causes the display 102 to display a radiation image based on the image data.

Each unit of the radiation imaging system SYS is not limited to the above arrangement, and the arrangement of each unit may be changed as needed in accordance with a purpose or the like. For example, the functions of two or more units such as the system controller 101 and the irradiation controller 103 may be implemented by one unit. In addition, for example, in this embodiment, the imaging unit 100 and the system controller 101 are exemplified as discrete units. However, this is not exhaustive. The imaging unit 100 may include some or all of the functions of the system controller 101, the display 102, and the irradiation controller 103 in addition to the existing functions of the imaging unit 100. Part of the function of a given unit may be implemented by another unit. For example, the imaging unit 100 may include the processor 130 of the system controller 101, which performs image processing. Alternatively, different functions of each unit may be implemented by different units. For example, the function as the processor 130 for performing image processing and the function as a controller for performing system control of the system controller 101 may be respectively implemented by different units.

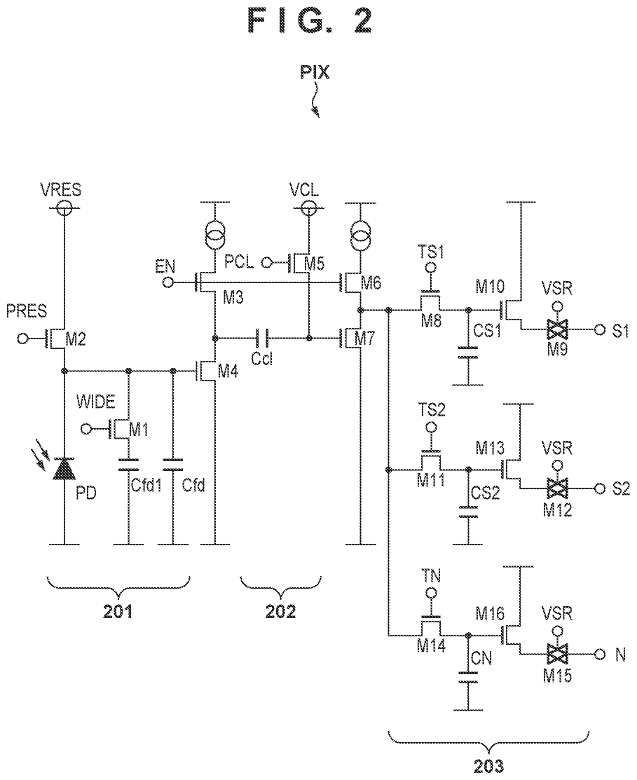

FIG. 2 shows an example of the circuit arrangement of one pixel PIX of a plurality of pixels provided in each sensor unit 120 of the sensor panel 105. Referring to FIG. 2, a photodiode PD is a photoelectric conversion element, which converts light generated by the above scintillator in accordance with incident radiation into an electrical signal. Accordingly, a converter 201 that generates a pixel signal corresponding to incident radiation is formed by including the scintillator and the photodiode PD as the conversion element. More specifically, the photodiode PD generates the amount of electric charges corresponding to the amount of light generated by the scintillator. This embodiment has exemplified the arrangement using the photodiode PD as the converter 201 for converting radiation into an electrical signal (electric charges), assuming the use of the sensor panel 105 using indirect type conversion elements as described above. However, this is not exhaustive. For example, direct type conversion elements for directly converting radiation into electrical signals may be used as the converter 201 for converting radiation into electrical signals.

In this embodiment, each pixel PIX includes capacitances Cfd and Cfd1, and a transistor M1 as a switch arranged between an output node of the converter 201 and the capacitance Cfd1. The capacitance Cfd is the capacitance of a floating diffusion (floating diffusion region) for accumulating the electric charges generated by the photodiode PD. The capacitance Cfd can include a parasitic capacitance parasitizing the photodiode PD. The transistor M1 is a switch element for switching the sensitivity of the pixel PIX with respect to radiation. The capacitance Cfd1 is a switching capacitor for switching the sensitivity of the pixel PIX, and is connected to the photodiode PD via the transistor M1. With this arrangement, in each pixel PIX, it is possible to change the capacitance value of the capacitance for accumulating the electric charges generated by the photodiode PD, thus switching the sensitivity of the pixel PIX with respect to radiation.

A transistor M2 is a reset switch for discharging the electric charges accumulated in the photodiode PD, the capacitance Cfd, and the capacitance Cfd1. A transistor M4 is an amplification MOS transistor (pixel amplifier) for operating as a source follower. A transistor M3 is a selection switch for rendering the transistor M4 operative.

A clamp circuit 202 for removing kTC noise generated by the converter 201 including the photodiode PD is arranged on the subsequent stage of the transistor M4. A capacitance Ccl is a clamp capacitance. A transistor M5 is a clamp switch for clamping. A transistor M7 is an amplification MOS transistor (pixel amplifier) operating as a source follower. A transistor M6 is a selection switch for rendering the transistor M7 operative.

A sampling circuit 203 provided with three sample/hold circuits is provided on the subsequent stage of the transistor M7. Transistor M8 and M11 are sample/hold switches constituting a sample/hold circuit for accumulating an optical signal that is an image pixel signal generated by the photodiode PD from light converted from radiation. Each of a capacitance CS1 and a capacitance CS2 is a holding capacitor for holding a sampled optical signal. A transistor M14 is a sample/hold switch constituting a sample/hold circuit for accumulating a reference voltage signal. A capacitance CN is a holding capacitor for holding a sampled reference signal. Each of transistors M10 and M13 is an amplification MOS transistor (pixel amplifier) for an optical signal, which operates as a source follower. Analog switches M9 and M12 are transfer switches for outputting the optical signals amplified by the transistors M10 and M13 to optical signal output circuits S1 and S2, respectively. A transistor M16 is an amplification MOS transistor (pixel amplifier) for a reference signal, which operates as a source follower. An analog switch M15 is a transfer switch for outputting a reference signal amplified by the transistor M16 to a reference signal output circuit N.

A signal EN is a control signal that is connected to the gates of the transistors M3 and M6 to control the operation states of the transistors M4 and M7. When the signal EN is set at high level, the transistors M4 and M7 are simultaneous rendered operative. A signal PRES is a control signal (reset signal) that is connected to the gate of the transistor M2 to control the operation state of the transistor M2. When the signal PRES is set at high level, the transistor M2 is turned on to discharge the electric charges accumulated in the photodiode PD and the capacitances Cfd and Cfd1. A signal PCL is a control signal that is connected to the gate of the transistor M5 to control the transistor M5. When the signal PCL is set at high level, the transistor M5 is turned on to set the capacitance Ccl at a reference voltage VCL. A signal TS1 is a control signal that is connected to the gate of the transistor M8 to control sampling/holding of an optical signal. The signals TS1 are set at high level to turn on the transistors M8 so as to collectively transfer optical signals to the capacitances CS1 via the transistors M7. Then, in all the pixels PIX, the signals TS1 are collectively set at low level to turn off the transistors M8 so as to complete sampling of optical signals to the capacitances CS1 of the sample/hold circuits. A signal TS2 is connected to the gate of the transistor M11 and operates in the same manner as the signal TS1, thereby sampling an optical signal to the capacitance CS2 of the sample/hold circuit. In this manner, in the plurality of pixels PIX, the sampling circuits 203 can simultaneously sample, with different sensitivities, the pixel signal (optical signal) generated by the photodiode PD. A signal TN is a control signal that is connected to the gate of the transistor M14 to control sampling/holding of a reference signal. When the signals TN are set at high level to turn on the transistors M14, reference signals are collectively transferred to capacitances CN via the transistors M7. In all the pixels, the signals TN are set at low level to turn off the transistors M14 so as to complete sampling of reference signals to the capacitances CN of the sample/hold circuits. The sampling circuits 203 can simultaneously sample the reference signals as well in the plurality of pixels PIX. After sampling/holding to the capacitances CS1, CS2, and CN, the transistors M8, M11, and M14 are turned off, and the capacitances CS1, CS2, and CN are disconnected from accumulation circuits on the preceding stage. Accordingly, optical signals and reference signals accumulated before next sampling can be nondestructively read out by rendering the respective analog switches M9, M12, and M15 conductive. That is, while the transistors M8, M11, and M14 are rendered non-conductive, held optical signals and reference signals can be read out at arbitrary timings.

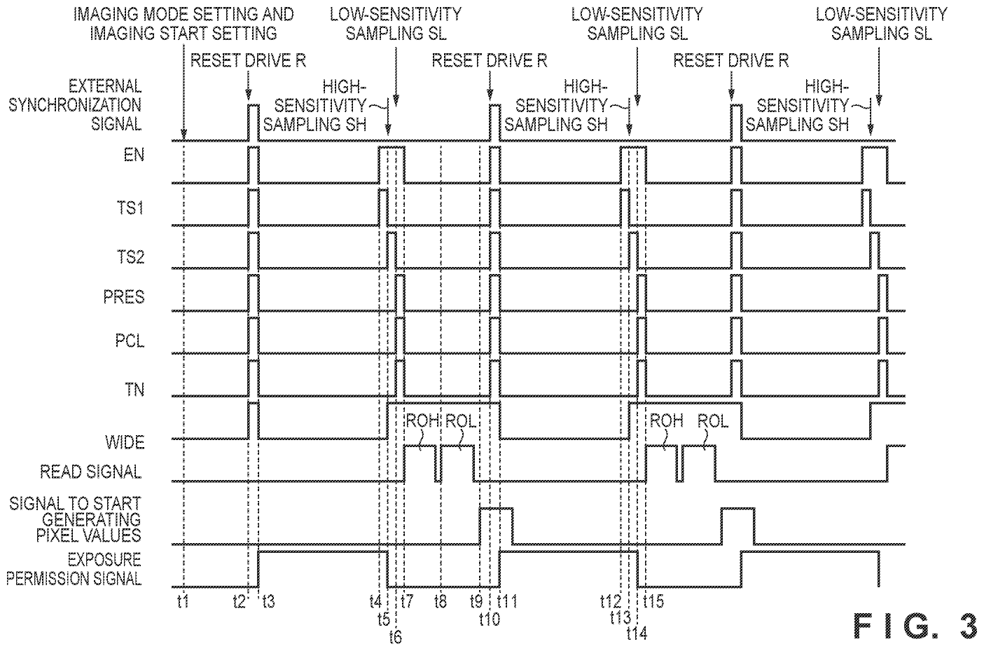

FIG. 3 is a timing chart showing an example of drive control upon capturing a moving image in the pixel PIX shown in FIG. 2. The timings of control signals until electric charges are sampled and held in the capacitances CS1, CS2, and CN upon capturing a moving image will be described below.

First of all, at time t1, a user makes settings of an imaging mode such as the sensitivity and accumulation time during imaging, and then sets the start of imaging. Then, at time t2, when the controller 109 detects that an external synchronization signal is set at high level, drive for imaging is started.

Here, reset drive R starting from time t2 will be described. The reset drive R is drive for resetting and clamping. First of all, at time t2, when the controller 109 detects that the synchronization signal 113 from the system controller 101 is set at high level, the controller 109 sets the signal EN at high level to turn on the transistors M4 and M7. Then, the controller 109 sets a signal WIDE and the signal PRES at high level to connect the photodiode PD to a reference voltage VRES while the transistor M1 is turned on. Then, the controller 109 sets the signal PCL at high level to turn on the transistor M5 as a clamp switch, and connects the transistor M7 side of the capacitance Ccl as a clamp capacitance to the reference voltage VCL. At the same time, the controller 109 sets the signals TS1, TS2, and TN at high level to turn on the transistors M8, M11, and M14.

At time t3, the controller 109 sets the signal WIDE at low level to turn off the transistor M1 so as to switch the converter 201 including the photodiode PD as the conversion element to the mode of detecting radiation with high sensitivity. In addition, the controller 109 sets the signal PRES at low level to finish the reset drive R.

At this time, the reset voltage VRES is set on the transistor M4 side of the capacitance Ccl. In addition, the transistor M1 side of the capacitance Cfd1 is also held at the reset voltage VRES to suppress the occurrence of an unstable voltage. The controller 109 also turns off the transistor M5. Electric charges corresponding to the differential voltage between the reference voltage VCL and the reference voltage VRES are accumulated in the capacitance Ccl, thereby finishing clamping. At the same time, the controller 109 also turns off the transistors M8, M11, and M14, and the reference signal obtained when the reference voltage VCL is set is held in the capacitances CS1, CS2, and CN. The influence of an after image is reduced by, before performing sampling, making uniform electric charges in the capacitances CS1 and CS2 each for holding an optical signal obtained by sampling, with predetermined sensitivity, the pixel signal generated by the photodiode PD, and electric charges in the capacitance CN for holding a reference signal.

At time t3, the reset drive R is finished, and the pixel PIX is set in an accumulating state. Accordingly, the controller 109 enables the exposure permission signal 114 to request radiation exposure. Time t3 can be regarded as a time to start an operation of accumulating pixel signals (electric charges) corresponding to applied radiation in each pixel PIX. The accumulation of electric charges in the photodiode PD and the capacitance Cfd of the floating diffusion is started in accordance with the start of irradiation with radiation by enabling the exposure permission signal 114. That is, the accumulation of signals corresponding to applied radiation with high sensitivity is started from time t3. In addition, the controller 109 sets the signal EN at low level to set the transistors M4 and M7 each constituting the pixel amplifier to be rendered inoperative.

The reset drive R is collectively performed for all the pixels PIX arranged in the radiation imaging apparatus 150. Subsequent reset drive R is also controlled at similar timings. In performing imaging for moving images or still images, in order to prevent image shifts caused by temporal switching shifts between pixels and scanning lines, the reset drive R can be performed for all the pixels PIX arranged in the radiation imaging apparatus 150 at the same timing in the same period. Thereafter, electric charges are accumulated by irradiation with radiation, and the signal charges generated by the photodiode PD of each pixel PIX are accumulated in the capacitance Cfd and the parasitic capacitance of the photodiode PD.

High-sensitivity sampling drive SH starting from time t4 will be described next. At time t4, the controller 109 sets the signal EN at high level to turn on the transistors M3 and M6. With this operation, the electric charges accumulated in the capacitance Cfd are charge/voltage-converted and output as a voltage to the capacitance Ccl by the transistor M4 operating as a source follower and constituting the pixel amplifier. Although an output from the transistor M4 contains reset noise, because the clamp circuit sets the transistor M7 side at the reference voltage VCL at the time of the reset drive R, the output is output as an optical signal from which reset noise is removed to the transistor M7 constituting the pixel amplifier. Then, the controller 109 sets the signal TS1, which controls sampling of an optical signal by irradiation with radiation, at high level to turn on the transistor M8. With this operation, in the pixels PIX arranged on the sensor panel 105, the optical signals (pixel signals) generated by the converters 201 are collectively transferred, via the transistors M7 each constituting the pixel amplifier, to the capacitances CS1 each for holding the signal obtained by sampling the optical signal with high sensitivity. At time t5, the controller 109 disables the exposure permission signal to stop the radiation exposure. In addition, the controller 109 sets the signal TS1 at low level to turn off the transistor M8. Thus, the signal sampled with high sensitivity is held in the capacitance CS1.

At time t5, the controller 109 sets the signal WIDE at high level to finish the sampling drive SH, and starts low-sensitivity sampling drive SL. When the signal WIDE is set at high level, the transistor M1 is turned on. When the transistor M1 as the switch for switching the sensitivity is turned on, the capacitance of the floating diffusion portion increases. Thus, the sampling circuit 203 can sample, with low sensitivity, the optical signal (pixel signal) generated by the converter 201.

Then, the controller 109 sets the signal TS2 at high level to turn on the transistor M11. With this operation, in the pixels PIX arranged on the sensor panel 105, the optical signals (pixel signals) generated by the converters 201 are collectively transferred, via the transistors M7 each constituting the pixel amplifier, to the capacitances CS2 each for holding the signal obtained by sampling the optical signal with low sensitivity. At time t6, the controller 109 sets the signal TS2 at low level to turn off the transistor M11. Thus, the signal sampled with low sensitivity is held in the capacitance CS2.

Then, the controller 109 sets the signal PRES at high level to turn on the transistor M2 so as to reset the capacitances Cfd and Cfd1 to the reference voltage VRES. The controller 109 then sets the signal PCL at high level. This accumulates, in the capacitance Ccl, electric charges with reset noise being superimposed on the differential voltage between the voltage VCL and the voltage VRES. In addition, the controller 109 sets the signal TN at high level to turn on the transistor M14 so as to transfer the reference signal set at the reference voltage VCL to the capacitance CN. Subsequently, at time t8, the controller 109 sets the signal TN at low level to turn off the transistor M14 so as to hold the reference signal in the capacitance CN. The controller 109 further sets the signals PRES, PCL, and EN at low level to finish the sampling drive SL.

With the operations described above, the sampling circuit 203 samples, with high sensitivity and low sensitivity, the optical signal (pixel signal) generated by the converter 201 in accordance with the incident radiation. The sampling drive SH for sampling the optical signals with high sensitivity and the sampling drive SL for sampling the optical signals with low sensitivity are performed collectively in all the pixels PIX arranged on the sensor panel 105. Thereafter, the subsequent sampling drive SH and sampling drive SL are also controlled at similar timings. After the sampling drive SL, if the controller 109 detects that the external synchronization signal is set at high level, the rest drive R is performed again from time t10, and accumulation of electric charges in the converter 201 is started for the next frame.

Scanning of a signal (to be sometimes referred to as an optical signal ROH hereinafter) sampled with high sensitivity, scanning of a signal (to be sometimes referred to as an optical signal ROL hereinafter) sampled with low sensitivity, and scanning of a reference signal are performed for each pixel PIX. When the controller 109 turns on the analog switches M9, M12, and M15, the voltages in the capacitances CS1, CS2, and CN are output to the corresponding column signal lines via the optical signal output circuits S1 and S2 and the reference signal output circuit N, respectively.

In the pixel PIX shown in FIG. 2, the timing of starting accumulation of electric charges in the converter 201 is each of times t3 and t11 at which the signal PCL is set at low level to complete clamping after the reset drive R shown in FIG. 3 is finished. The timing of finishing the accumulation of the electric charges in the converter 201 is each of times t5 and t13 at which the signal TS1 is set at low level, the exposure permission signal 114 is disabled, and the optical signal ROH is sampled.

After the sampling drive SL is finished, from time t7, the controller 109 performs readout processing of reading out the optical signal ROH from each pixel PIX. When the readout processing of the optical signal ROH is finished, from time t8, the controller 109 then performs readout processing of reading out the optical signal ROL from each pixel PIX.

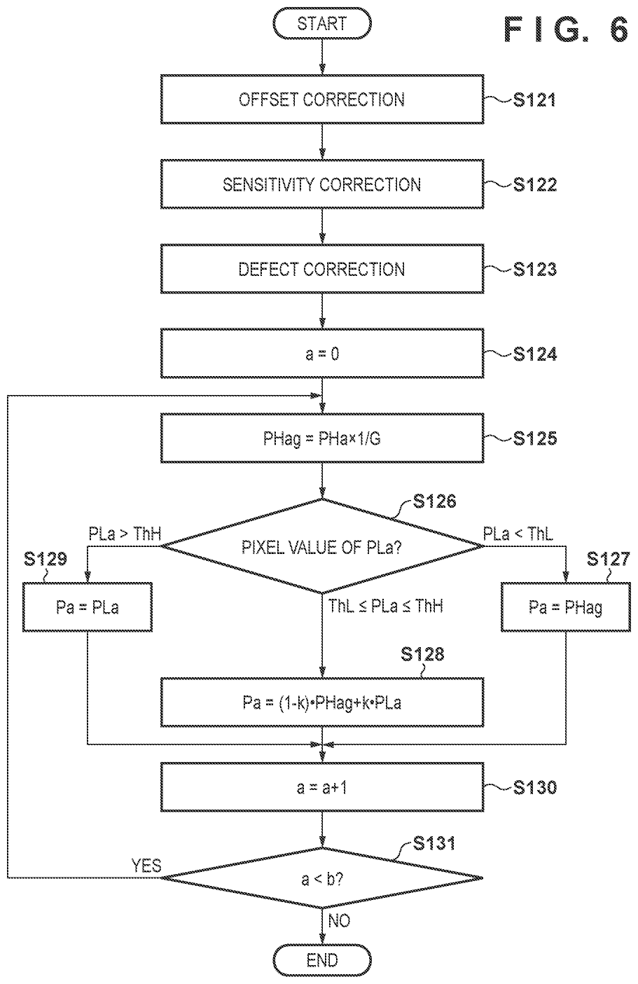

When reading out of the optical signal ROL is finished, at time t9, the controller 109 transmits a signal indicating to start processing of the optical signals ROH and ROL to the processor 130 of the system controller 101 via the control interface 110. In accordance with this signal, the processor 130 starts processing of generating a pixel value for each pixel PIX of the plurality of pixels PIX arranged on the sensor panel 105 of the imaging unit 100. The processing in the processor 130 will be described later with reference to FIG. 6.

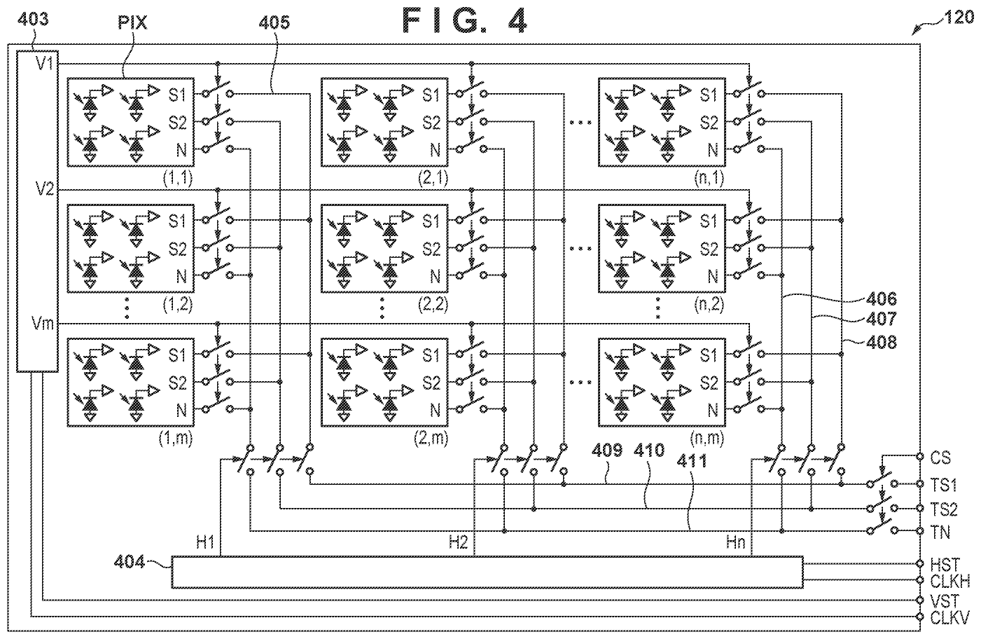

FIG. 4 is a view schematically showing an example of the arrangement of the internal structure of the sensor unit 120. Each sensor unit 120 includes a chip select terminal CS, an optical signal output terminal TS1, an optical signal output terminal TS2, a reference signal output terminal TN, a vertical scanning circuit start signal terminal VST, a vertical scanning circuit clock terminal CLKV, a horizontal scanning circuit start signal terminal HST, and a horizontal scanning circuit clock terminal CLKH. On each sensor unit 120, m pixels PIX and n pixels PIX are two-dimensionally arrayed in the column and row directions, respectively. A vertical scanning circuit 403 selects the pixels PIX arranged in the row direction for each row, and sequentially scans pixel groups in the vertical direction as the sub-scanning direction in synchronism with the vertical scanning clock CLKV. The vertical scanning circuit 403 can be formed from, for example, a shift register. A horizontal scanning circuit 404 sequentially selects the column signal lines of the pixels PIX in the column direction as the main scanning direction selected by the vertical scanning circuit 403 pixel by pixel in synchronism with the horizontal scanning clock CLKH. When a row signal line 405 connected to the vertical scanning circuit 403 is enabled, each pixel PIX outputs sampled optical signals and reference signal to column signal lines 406, 407, and 408, respectively. When the horizontal scanning circuit 404 sequentially selects signals respectively output to the column signal lines 406, 407, and 408, signals of the respective pixels PIX are sequentially output to analog output lines 409, 410, and 411. As has been described above, the sensor unit 120 selects the pixel PIX by a switching operation based on an XY address scheme using the vertical scanning circuit 403 and the horizontal scanning circuit 404. Optical signals and a reference signal from each pixel PIX are output from the optical signal output terminal TS1, the optical signal output terminal TS2, and the reference signal output terminal TN via the column signal lines 406, 407, and 408 and the analog output lines 409, 410, and 411.

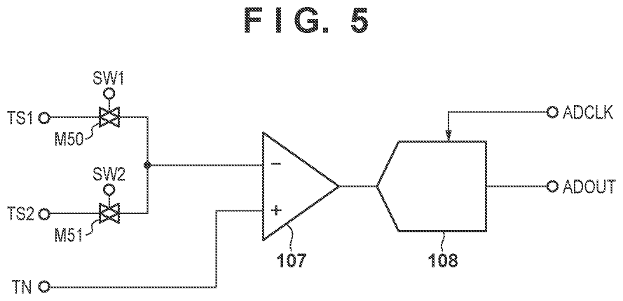

FIG. 5 is a view showing an arrangement example of the readout circuit 106 including the differential amplifier 107 and the A/D converter 108 which A/D-convert optical signals and a reference signal output from each pixel PIX. Outputs from the optical signal output terminals TS1 and TS2 are respectively connected to an input switch M50 and an input switch M51. The input switch M50 operates based on a signal SW1, and the input switch M51 operates based on a signal SW2. In order to prevent the destruction of signals output from each pixel PIX or the destruction of elements, the signals SW1 and SW2 are controlled by the controller 109 so as not to simultaneously turned on the input switches M50 and M51.

Even when, for example, optical signals (an optical signal sampled and held in the capacitance CS1 is sometimes referred to as an optical signal 1, and an optical signal sampled and held in the capacitance CS2 is sometimes referred to as an optical signal 2 hereinafter) and a reference signal sampled and held in the capacitances CS1, CS2, and CN of each pixel PIX, respectively, are simultaneously output, the controller 109 first controls the signal SW1 at high level and the signal SW2 at low level. Then, the optical signals 1 and reference signals are sequentially read out from a pixel PIX (1, 1) to a pixel PIX (n, m) shown in FIG. 4. Subsequently, the controller 109 may control the signal SW1 at low level and the signal SW2 at high level to sequentially read out the optical signals 2 and reference signals from the pixel PIX (1, 1) to the pixel PIX (n, m).

Alternatively, for example, the controller 109 controls the signal SW1 at high level and the signal SW2 at low level to sequentially read out the optical signals 1 and reference signals from the pixel PIX (1, 1) to a pixel PIX (n, 1). Then, the controller 109 controls the signal SW1 at low level and the signal SW2 at high level to sequentially read out the optical signals 2 and reference signals from the pixel PIX (1, 1) to the pixel PIX (n, 1). Then, the controller 109 supplies the vertical scanning clock CLKV to the vertical scanning circuit 403 to scan one pixel in the sub-scanning direction, thereby sequentially selecting pixels from a pixel PIX (1, 2) to a pixel PIX (n, 2). The controller 109 again controls the signal SW1 at high level and the signal SW2 at low level to sequentially read out the optical signals 1 and reference signals from the pixel PIX (1, 2) to the pixel PIX (n, 2). Then, the controller 109 controls the signal SW1 at low level and the signal SW2 at high level to sequentially read out the optical signals 2 and reference signals from the pixel PIX (1, 2) to the pixel PIX (n, 2). In this manner, the controller 109 may control the signals SW1 and SW2 for each row to sequentially read out the optical signals and reference signals from the pixel PIX (1, 1) to the pixel PIX (n, m).

The differential amplifier 107 receives an optical signal acquired with high or low sensitivity at the negative side input and a reference signal at the positive side input. Subtracting an optical signal from a reference signal by using the differential amplifier 107 makes it possible to remove fixed pattern noise (FPN) caused by process variations or the like between the pixel amplifiers in each pixel PIX. An output from the differential amplifier 107 is input to the A/D converter 108. The A/D converter 108 receives a clock signal from a signal ADCLK, and outputs a digital optical signal ADOUT having undergone A/D conversion at the timing when the signal ADCLK is switched to high level to the controller 109 for each sensor unit 120.

The processing performed by the processor 130 to suppress deterioration of the linearity of an A/D-converted signal value with respect to the incident dose in a low dose region while ensuring high dynamic range will be described next. The optical signals ADOUT transmitted to the controller 109 are transmitted to the processor 130 of the system controller 101 via the image data interface 111 in the order of reading out by the readout circuits 106, and undergo processing for improving the linearity. FIG. 6 is a flowchart for explaining the processing for improving the linearity.

The processor 130 receives, from the controller 109, signals ROH′ and ROL′ obtained by performing correction using the reference signal and A/D conversion on the optical signals ROH and ROL, which have been obtained by sampling the optical signal generated by the converter 201 by the sampling circuit 203 with high sensitivity and low sensitivity as described above. The signals ROH′ and ROL′ correspond to the optical signals ADOUT described above. Further, as has been described above, when a signal indicating to start the processing is received from the controller 109, the processor 130 starts processing of generating a pixel value for each pixel PIX of the plurality of pixels arranged on the sensor panel 105 of the imaging unit 100.

First of all, in order to correct the characteristics of the sensor panel 105 and the characteristics for each sensor unit 120, the processor 130 performs offset correction (S121), sensitivity correction (S122), and defect correction (S123). At this time, the correlation between the signal value of the signal output from each pixel PIX and the signal value of the signal output from the pixel PIX in the vicinity thereof is maintained.

Next, in step S124, the processor 130 initializes a variable a representing the position of the pixel PIX in the sensor panel 105 to “0”. For example, the pixel arranged at the coordinates (1, 1) in FIG. 4 may be the pixel PIX with a=0. For the sensor panel 105 having the arrangement shown in FIGS. 1 and 4, since n×m pixels PIX are arranged in the sensor unit 120, a maximum value b of the variable a is given as b=(n×m×7×2)−1.

Here, let PLa be the signal value of the signal ROL′ corresponding to the low-sensitivity optical signal ROL when the pixel position is represented by “a”, and PHa be the signal value of the signal ROH′ corresponding to the high-sensitivity optical signal ROH. Further, let G be the sensitivity ratio between the sampling with high sensitivity and the sampling with low sensitivity.

After the initialization of the position of the pixel PIX in step S124, in step S125, the processor 130 multiplies the signal value PHa of the signal ROH′ sampled with high sensitivity by 1/G, thereby obtaining a sensitivity adjustment value PHag.

After the sensitivity adjustment value PHag is generated, the process transitions to step S126 and, in accordance with the magnitude of the signal value PLa based on the optical signal ROL sampled with low sensitivity from the converter 201, the processor 130 selects a generation method of a pixel value Pa of the pixel PIX at the position a. In this embodiment, two threshold values are set. Here, the two threshold values are a threshold value ThL and a threshold value ThH larger than the threshold value ThL (ThH>ThL). Here, a case will be described in which the digital image data ADOUT having undergone A/D conversion by the A/D converter 108 is 16-bit data.

If the low-sensitivity signal value PLa is smaller than the threshold value ThL, the processor 130 sets, as the pixel value Pa of the pixel PIX at the pixel position “a”, the sensitivity adjustment value PHag obtained by adjusting, in accordance with the sensitivity ratio, the pixel value PHa obtained by sampling with high sensitivity (step S127). That is, if the signal value PLa obtained by sampling the optical signal (pixel signal) with low sensitivity by the sampling circuit 203 is smaller than the threshold value ThL, the processor 130 generates the pixel value Pa of the pixel based on the signal value PHa obtained by sampling the optical signal with high sensitivity by the sampling circuit 203.

If the low-sensitivity signal value PLa is larger than the threshold value ThH, the processor 130 sets, as the pixel value Pa of the pixel PIX at the pixel position “a”, the signal value PLa obtained by sampling with low sensitivity (step S129). That is, if the signal value PLa obtained by sampling the optical signal with low sensitivity by the sampling circuit 203 exceeds the threshold value ThH larger than the threshold value ThL, the processor 130 generates the pixel value Pa of the pixel based on the signal value PLa obtained by sampling the optical signal with low sensitivity by the sampling circuit 203.

Further, if the low-sensitivity signal value PLa is equal to or larger than the threshold value ThL (equal to or larger than the first threshold value) and equal to or smaller than the threshold value ThH (equal to or smaller than the second threshold value), the processor 130 generates the pixel value Pa of the pixel based on the signal value PHa (sensitivity adjustment value PHag) obtained by sampling the optical signal with high sensitivity by the sampling circuit 203 and the signal value PLa obtained by sampling with low sensitivity (step S128). At this time, the processor 130 may generate the pixel value Pa of the pixel based on a weighted average value of the signal value PHa (sensitivity adjustment value PHag) and the signal value PLa. More specifically, the processor 130 generates the pixel value Pa using a predetermined weighting coefficient k and following equation (1):

Pa=(1−k)×PHag+k×PLa (1)

The weighting coefficient k may be generated using, for example, following equation (2):

k=(PLa−ThL)/(ThH−ThL) (2)

With this, the pixel value Pa obtained by combining the signal value PLa and the signal value PHa (sensitivity adjustment value PHag) with predetermined weighting is generated. As a result, it is possible to smooth the boundary between an image based on the pixel value generated from the signal obtained by sampling with low sensitivity and an image based on the pixel value generated from the signal obtained by sampling with high sensitivity.

When the pixel value Pa of one pixel PIX is generated, the processor 130 transitions to step S130, and adds 1 to the variable a representing the pixel position. Then, if the value of the variable a is smaller than the maximum value b (YES in step S131), the process returns to step S125, and the processor 130 starts to generate the pixel value Pa for the next pixel position. If the value of the variable a is equal to or larger than the maximum value b (NO in step S131), the process advances to step S132, and the processor 130 terminates the processing of generating the pixel value Pa for each pixel PIX.

When the converter 201 is set at low sensitivity to implement high dynamic range, the signal value of the signal output from the converter 201 in a region where the incident radiation dose is small in the sensor panel 105 is relatively small as compared to a case of setting the converter at high sensitivity. Therefore, an influence of 1/f noise or the like increases, and the linearity of the A/D-converted signal value with respect to the incident dose can deteriorate. To prevent this, among the pixels PIX arranged on the sensor panel 105, for the pixel PIX in which the signal value PLa obtained by sampling with low sensitivity is smaller than the predetermined threshold value ThL, the signal value PLa is replaced with the sensitivity adjustment value PHag based on the signal value PHa obtained by sampling with high sensitivity. With this, the radiation imaging apparatus 150 in this embodiment can generate an image with improved linearity in a low dose region.

The threshold value ThL and the threshold value ThH may be changeable in accordance with the radiation irradiation condition or the like. For example, the processor 130 may have a function of changing the values of the threshold values ThL and ThH. For example, the processor 130 may change the threshold value ThL and the threshold value ThH based on at least one of a dose to be applied during irradiation with radiation or an accumulation time, which are set by a user. Further, for example, the processor 130 may change the threshold value ThL and the threshold value ThH based on a signal value (to be sometimes referred to as a saturation value hereinafter) at which the optical signal (pixel signal) is saturated in sampling with high sensitivity, which is decided by the capacitance value of the capacitance Cfd or the like. For example, the threshold value ThL and the threshold value ThH may be decided based on the value obtained by dividing the saturation value by the sensitivity ratio G. For example, it may be set that threshold value ThL=(saturation value/G)×0.8 and threshold value ThH=(saturation value/G)×0.9. Further, the relationship (linearity) between the incident radiation dose and the output signal value in sampling with low sensitivity may be measured in advance, and the threshold value ThL and the threshold value ThH may be decided based on the linearity.

As has been described above, when the imaging unit 100 of the radiation imaging apparatus 150 includes the memory 115, the memory 115 may store the threshold value ThL and the threshold value ThH corresponding to the radiation irradiation condition set by the user. In this case, the processor 130 may read out, from the memory 115, the threshold value ThL and the threshold value ThH in accordance with the irradiation condition, and use them to generate the pixel value Pa.

In this embodiment, the processor 130 is arranged outside the imaging unit 100. However, as has been described above, the processor 130 may be arranged in the imaging unit 100, or the processing in the processor 130 may be performed by the controller 109.

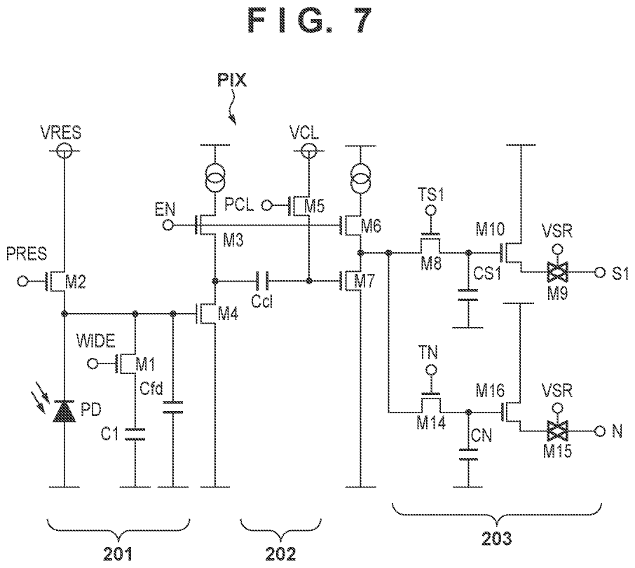

FIG. 7 shows a modification of the pixel PIX shown in FIG. 2. This is different from the arrangement shown in FIG. 2 in that the sampling circuit 203 is formed from two sample/hold circuits. More specifically, the number of circuits for sampling and holding the optical signal corresponding to the radiation is changed from two to one, and this circuit is formed from the transistor M8, the capacitance CS1 as the holding capacitor for holding an optical signal, the transistor M10, and the analog switch M9.

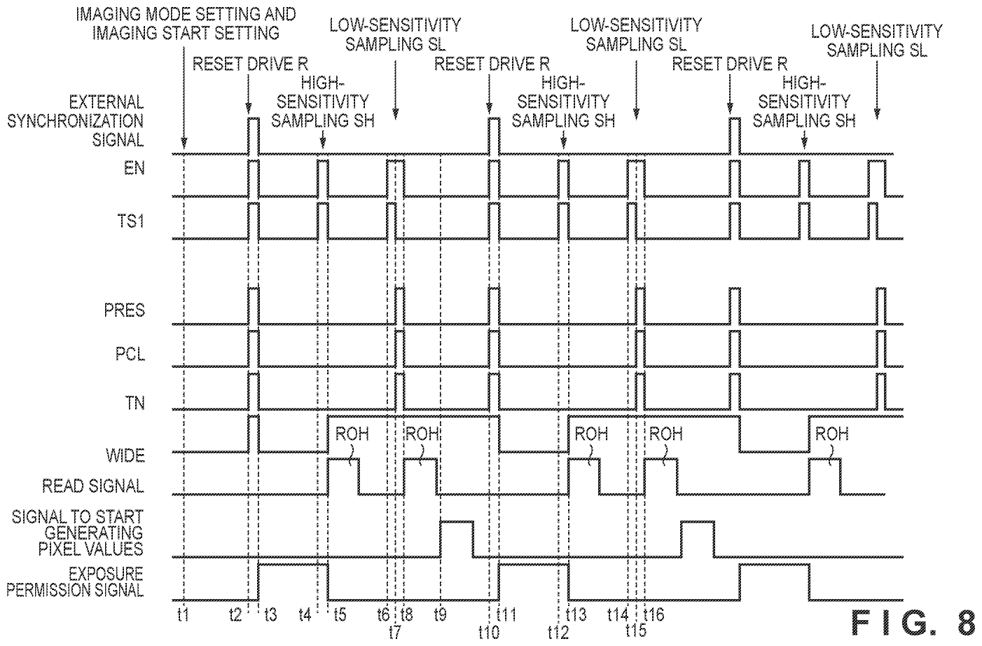

FIG. 8 is a timing chart showing an example of drive control upon capturing a moving image in the pixel PIX shown in FIG. 7. The timings of control signals until electric charges are sampled and held in the capacitances CS1 and CN upon capturing a moving image will be described below.

First of all, at time t1, a user makes settings of an imaging mode such as the sensitivity and accumulation time during imaging, and then sets the start of imaging. Then, at time t2, when the controller 109 detects that an external synchronization signal is set at high level, drive for imaging is started.

Here, reset drive R starting from time t2 will be described. The reset drive R is drive for resetting and clamping. First of all, at time t2, when the controller 109 detects that the synchronization signal 113 from the system controller 101 is set at high level, the controller 109 sets the signal EN at high level to turn on the transistors M4 and M7. Then, the controller 109 sets the signal WIDE and the signal PRES at high level to connect the photodiode PD to the reference voltage VRES while the transistor M1 is turned on. Then, the controller 109 sets the signal PCL at high level to turn on the transistor M5 as a clamp switch, and connects the transistor M7 side of the capacitance Ccl as a clamp capacitance to the reference voltage VCL. At the same time, the controller 109 sets the signals TS1 and TN at high level to turn on the transistors M8 and M14.

At time t3, the controller 109 sets the signal WIDE at low level to turn off the transistor M1 so as to switch the converter 201 including the photodiode PD to the mode of detecting radiation with high sensitivity. In addition, the controller 109 sets the signal PRES at low level to finish the reset drive R.

At this time, the reset voltage VRES is set on the transistor M4 side of the capacitance Ccl. In addition, the transistor M1 side of the capacitance Cfd1 is also held at the reset voltage VRES to suppress the occurrence of an unstable voltage. The controller 109 also turns off the transistor M5. Electric charges corresponding to the differential voltage between the reference voltage VCL and the reference voltage VRES are accumulated in the capacitance Ccl, thereby finishing clamping. At the same time, the controller 109 also turns off the transistors M8 and M14, and the reference voltage signal obtained when the reference voltage VCL is set is held in the capacitances CS1 and CN. The influence of an after image is reduced by, before performing sampling, making uniform electric charges in the capacitance CS1 and the capacitance CN.

At time t3, the reset drive R is finished, and the pixel PIX is set in an accumulating state. Accordingly, the controller 109 enables the exposure permission signal 114 to request radiation exposure. Time t3 can be regarded as a time to start an operation of accumulating pixel signals (electric charges) corresponding to applied radiation in each pixel PIX. The accumulation of electric charges in the photodiode PD and the capacitance Cfd of the floating diffusion is started in accordance with the start of irradiation with radiation by enabling the exposure permission signal 114. That is, the accumulation of signals corresponding to applied radiation with high sensitivity is started from time t3. In addition, the controller 109 sets the signal EN at low level to set the transistors M4 and M7 each constituting the pixel amplifier to be rendered inoperative.

The reset drive R is collectively performed for all the pixels PIX arranged in the radiation imaging apparatus 150. Subsequent reset drive R is also controlled at similar timings. In performing imaging for moving images or still images, in order to prevent image shifts caused by temporal switching shifts between pixels and scanning lines, the reset drive R can be performed for all the pixels PIX arranged in the radiation imaging apparatus 150 at the same timing in the same period. Thereafter, electric charges are accumulated by irradiation with radiation, and the signal charges generated by the photodiode PD of each pixel PIX are accumulated in the capacitance Cfd and the parasitic capacitance of the photodiode PD.

High-sensitivity sampling drive SH starting from time t4 will be described next. At time t4, the controller 109 sets the signal EN at high level to turn on the transistors M3 and M6. With this operation, the electric charges accumulated in the capacitance Cfd are charge/voltage-converted and output as a voltage to the capacitance Ccl by the transistor M4 operating as a source follower and constituting the pixel amplifier. Although an output from the transistor M4 contains reset noise, because the clamp circuit sets the transistor M7 side at the reference voltage VCL at the time of the reset drive R, the output is output as an optical signal from which reset noise is removed to the transistor M7 constituting the pixel amplifier. Then, the controller 109 sets the signal TS1, which controls sampling of an optical signal by irradiation with radiation, at high level to turn on the transistor M8. With this operation, in the pixels PIX arranged on the sensor panel 105, the optical signals (pixel signals) generated by the converters 201 are collectively transferred, via the transistors M7 each constituting the pixel amplifier, to the capacitances CS1 each for holding the signal obtained by sampling the optical signal with high sensitivity. At time t5, the controller 109 disables the exposure permission signal to stop the radiation exposure. In addition, the controller 109 sets the signal TS1 at low level to turn off the transistor M8. Thus, the signal sampled with high sensitivity is held in the capacitance CS1.

At time t5, the controller 109 sets the signal WIDE at high level to finish the sampling drive SH. When the signal WIDE is set at high level, the transistor M1 is turned on. When the transistor M1 as the switch for switching the sensitivity is turned on, the capacitance of the floating diffusion portion increases. Thus, the sampling circuit 203 can sample, with low sensitivity, the optical signal (pixel signal) generated by the converter 201. Then, the processing is performed for reading out the signal ROH sampled and held in the capacitance CS1 of the sampling circuit 203 with high sensitivity after finishing the sampling drive SH.

After reading out the signals ROH from the respective pixels PIX, at time t6, the controller 109 sets the signal EN at high level to turn on the transistor M3 and the transistor M6. Then, the controller 109 sets the signal TS1 at high level to turn on the transistor M8. With this operation, in the pixels PIX arranged on the sensor panel 105, the optical signals (pixel signals) generated by the converters 201 are collectively transferred, via the transistors M7 each constituting the pixel amplifier, to the capacitances CS1. At time t7, the controller 109 sets the signal TS1 at low level to turn off the transistor M8. Thus, the signal sampled with low sensitivity is held in the capacitance CS1.

Then, the controller 109 sets the signal PRES at high level to turn on the transistor M2 so as to reset the capacitances Cfd and Cfd1 to the reference voltage VRES. The controller 109 then sets the signal PCL at high level. This accumulates, in the capacitance Ccl, electric charges with reset noise being superimposed on the differential voltage between the voltage VCL and the voltage VRES. In addition, the controller 109 sets the signal TN at high level to turn on the transistor M14 so as to transfer the reference signal set at the reference voltage VCL to the capacitance CN. Subsequently, at time t8, the controller 109 sets the signal TN at low level to turn off the transistor M14 so as to hold the reference signal in the capacitance CN. The controller 109 further sets the signal PRES at low level to finish the resetting. In addition, the controller 109 sets the signal PCL at low level and the signal EN at low level, to finish the low-sensitivity sampling drive SL. After the sampling drive SL is finished, processing is performed for reading out the signal ROL sampled and held in the capacitance CS1 of the sampling circuit 203 with low sensitivity.

The sampling drive SH for sampling the optical signals with high sensitivity and the sampling drive SL for sampling the optical signals with low sensitivity are performed collectively in all the pixels PIX arranged on the sensor panel 105. Thereafter, the subsequent sampling drive SH and sampling drive SL are also controlled at similar timings. After the sampling drive SL, if the controller 109 detects that the external synchronization signal is set at high level, the rest drive R is performed again from time t10, and accumulation of electric charges in the converter 201 is started for the next frame.

Scanning of the signal ROH sampled with high sensitivity and scanning of the reference signal are performed for each pixel PIX. When the controller 109 turns on the analog switches M9 and M15, the voltages in the capacitances CS1 and CN are output to the corresponding column signal lines via the transistors M10 and M16 functioning as the pixel amplifiers, and the optical signal output circuit S1 and the reference signal output circuit N, respectively.

In the pixel PIX shown in FIG. 7, the timing of starting accumulation of electric charges in the converter 201 is each of times t3 and t11 at which the signal PCL is set at low level to complete clamping after the reset drive R shown in FIG. 8 is finished. The timing of finishing the accumulation of the electric charges in the converter 201 is each of times t5 and t13 at which the signal TS1 is set at low level and the optical signal ROH is sampled.

When reading out of the optical signal ROL sampled with low sensitivity is finished, at time t9, the controller 109 transmits a signal indicating to start processing of the optical signals ROH and ROL to the processor 130 of the system controller 101 via the control interface 110. In accordance with this signal, the processor 130 starts processing of generating a pixel value for each pixel PIX of the plurality of pixels PIX arranged on the sensor panel 105 of the imaging unit 100. The processing of generating a pixel value may be similar to the method illustrated in FIG. 6 described above, so that a description thereof will be omitted here. Also in the radiation imaging apparatus 150 including the pixel PIX having the arrangement shown in FIG. 7, by performing the operations shown in FIG. 8, it is possible to generate an image with improved linearity in a low dose region as in the case described above.

In this embodiment, sensitivity is switched between two stages of low sensitivity and high sensitivity. However, a switching capacitor for switching additional sensitivity and a switch between the output node of the photodiode PD and the additional switching capacitor may be further provided, and sensitivity may be switched among three or more stages. That is, the sampling circuit 203 may be capable of sampling the optical signal (pixel signal) with third sensitivity (for example, intermediate sensitivity, higher sensitivity, lower sensitivity (higher dynamic range), or the like) different from low sensitivity and high sensitivity. For example, a case in which sensitivity is switched among three stages of low sensitivity, intermediate sensitivity, and high sensitivity is also applicable. In this case, for example, as a combination of sensitivities, not only a combination of low sensitivity and high sensitivity but also a combination of low sensitivity and intermediate sensitivity and a combination of intermediate sensitivity and high sensitivity are applicable. Further, by increasing the number of switching capacitors or combining a plurality of kinds of capacitance values of the switching capacitor, the radiation imaging apparatus 150 may be configured to be capable of sampling a signal with four or more kinds of sensitivities.

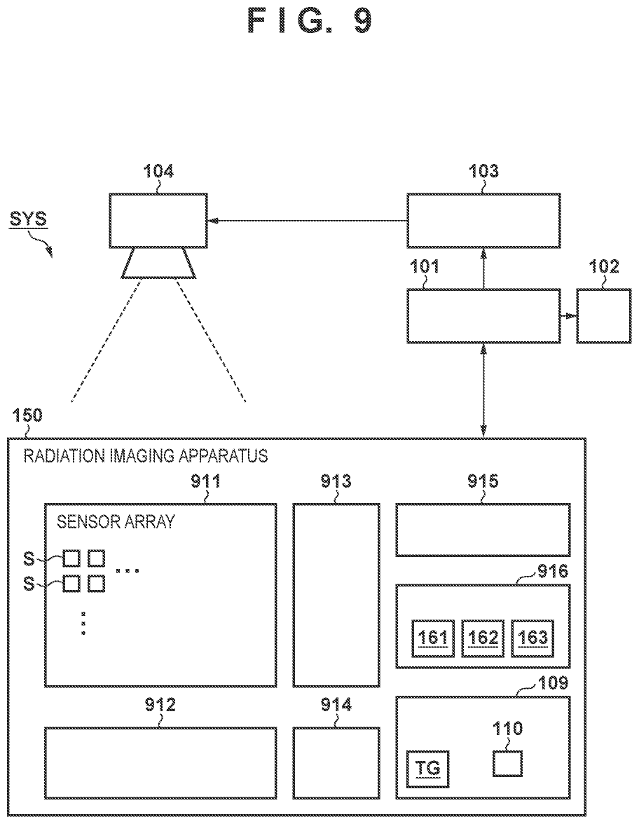

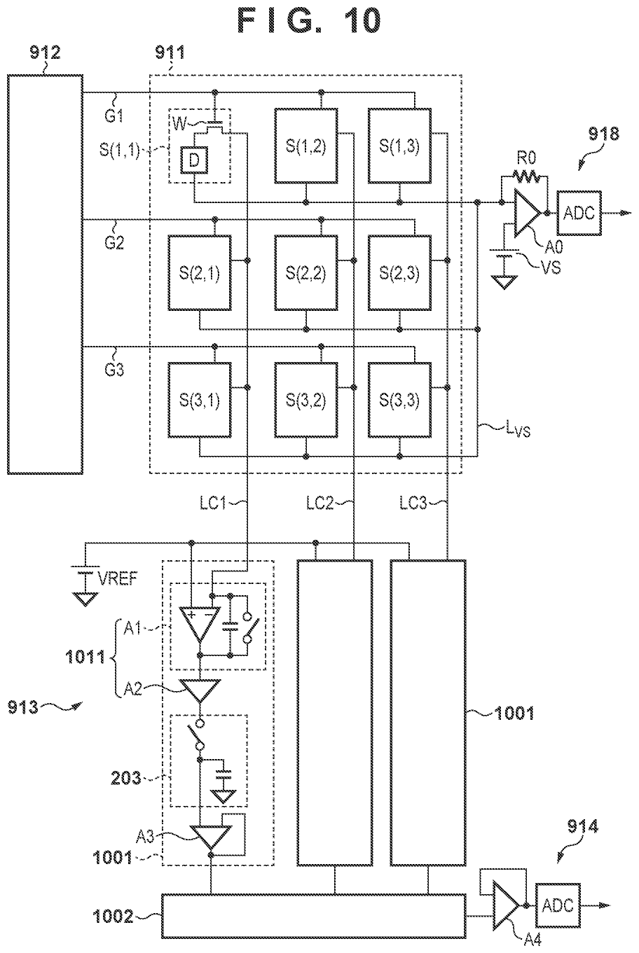

With reference to FIGS. 9 and 10, the arrangement of a radiation imaging apparatus and a drive method therefor according to this embodiment will be described. In the first embodiment described above, an example has been described in which the switching capacitor (capacitance Cfd1) for switching the sensitivity upon detecting radiation is arranged in the pixel PIX. However, the sensitivity for detecting radiation may be changed outside the pixel PIX. FIG. 9 is a system block diagram showing an example of the overall configuration of a radiation imaging system SYS including a radiation imaging apparatus 150 according to this embodiment.

The radiation imaging system SYS includes the radiation imaging apparatus 150, a radiation source 104, an irradiation controller 103, and a system controller 101. The system controller 101 communicates with the radiation imaging apparatus 150 based on an imaging condition input by a user such as a doctor or a radiologist, thereby controlling the radiation imaging apparatus 150. Further, the system controller 101 drives the radiation source 104 via the irradiation controller 103. The radiation source 104 generates radiation in accordance with a control signal from the irradiation controller 103. The radiation imaging system SYS may further include a display 102 that displays a radiation image based on image data output from the system controller 101.

The radiation imaging apparatus 150 includes a pixel array 911, a driver 912, a readout circuit 913, an output circuit 914, a notification unit 915, a power supply circuit 916, and a controller 109. In the pixel array 911, a plurality of pixels S each including a converter D for generating a pixel signal corresponding to incident radiation are arranged in a matrix (so as to form a plurality of rows and a plurality of columns). The pixel array 911 may be a combination of a plurality of dividable sensor units 120 like the sensor panel 105 shown in FIG. 1 described above, or the plurality of pixels S may be arranged in a matrix on one substrate.

The driver 912 is a vertical scanning circuit for driving the plurality of pixels S for each row, and can be formed from, for example, a shift register or the like. For example, the driver 912 can reset (initialize) the pixel S and cause the pixel S to output a pixel signal. Although details will be described later, the readout circuit 913 can be formed by including, for example, an amplifier and the like. Via a plurality of column signal lines LC to which pixel signals are transferred from the converters D of the pixels S, of the plurality of pixels S, arranged in a column direction, the pixel signals transferred to the column signal lines LC are read out for each column. The output circuit 914 outputs, as image data for one frame, a group of pixel signals read out by the readout circuit 913. The notification unit 915 is, for example, a light source, a display, or the like, and notifies a user of the state (for example, operation mode) of the radiation imaging apparatus 150.

The power supply circuit 916 generates, from a power supply voltage supplied from the outside, a voltage to be supplied to each component in the radiation imaging apparatus 150. More specifically, each component in the radiation imaging apparatus 150 is formed from one or more IC chips (integrated circuit chips (semiconductor chips)) and the like, and the power supply circuit 916 is a power supply IC that generates a voltage to be supplied to the components such as the IC chips. Typically, the power supply circuit 916 includes an AD-DC converter and one or more DC-DC converters. Although shown as a single unit in FIG. 9, the power supply circuit 916 may be formed from a plurality of power supply ICs. In this embodiment, the power supply circuit 916 includes switching-type voltage regulators 161 and 162 used for the DC-DC converters, thereby generating a desired constant voltage. The voltage regulator may be referred to as a switching regulator.

Although details will be described later, the power supply circuit 916 further includes a signal generator 163 that generates a clock signal for performing switching control of the voltage regulators 161 and 162. For the sake of descriptive convenience, the single signal generator 163 is shown here, but one signal generator 163 may be provided for each of the voltage regulators 161 and 162. In addition, for the sake of descriptive convenience, the signal generator 163 is described here as a separate unit from the voltage regulators 161 and 162, but the signal generator 163 may be included in each of the voltage regulators 161 and 162. In this case, each of the voltage regulators 161 and 162 can generate a clock signal by itself.

The controller 109 includes a timing generator TG and generates, based on a reference clock signal, a control signal for synchronous control of the respective components in the radiation imaging apparatus 150. The controller 109 can also function as a processor and, for example, can perform data processing such as correction processing for image data read out by the readout circuit 913 and the output circuit 914. Here, different from the first embodiment described above, a description will be given assuming that a processor 130 that generates a pixel value of each pixel S is arranged in the controller 109.

The controller 109 may be, for example, an integrated circuit or a device (for example, a PLD (Programmable Logic Device) such as an FPGA (Field Programmable Gate Array)) in which the respective functions described in this specification can be programmed, or may be an arithmetic apparatus such as an MPU (Micro Processing Unit) or a DSP (Digital Signal Processor), a dedicated integrated circuit ((ASIC (Application Specific Integrated Circuit)), or the like for implementing the respective functions. In addition, the respective functions may be implemented on software by a personal computer including a CPU (Central Processing Unit) and a memory and storing predetermined programs, or the like. That is, the functions of the controller 109 may be implemented by hardware and/or software.

It is needless to say that the configuration example of the radiation imaging system SYS is not limited to this example, and functions of some of the respective components forming the radiation imaging system SYS may be included in another unit, or a unit having another function may be added. For example, some of the functions of the radiation imaging apparatus 150 may be implemented by the system controller 101, and vice versa. For example, in the configuration shown in FIG. 9, the system controller 101 and the controller 109 are individually shown, but some or all of the functions thereof may be implemented by a single unit.

FIG. 10 illustrates the arrangement of the pixel array 911, the driver 912, the readout circuit 913, and the output circuit 914 in the arrangement of the radiation imaging apparatus 150. For the sake of descriptive convenience, the pixel array 911 on which the plurality of pixels S are arrayed in 3 rows×3 columns is illustrated here, but the actual numbers of rows and columns are larger than in this example. For example, for the 17-inch pixel array 911, the pixels S are arrayed in about 3,000 rows×3,000 columns pixels. Note that in FIG. 10, the pixel S in the mth row and the nth column is represented as “S (m, n)”. For example, a pixel S (1, 1) is located in the first row and the first column in the pixel array 911.

In this embodiment, a scintillator (not shown) for converting radiation into light is arranged above the pixel array 911, and the pixel S outputs a pixel signal based on the light (scintillation light) converted by the scintillator. More specifically, the pixel S includes the converter D and a switch element W connected to the converter D. In this embodiment, an MIS sensor is used as the converter D, but another photoelectric conversion element such as a PIN sensor may be used. In addition, a thin film transistor (TFT (Thin Film Transistor)) is used as the switch element W, but a transistor or switch element having another structure may be used. On the opposite side of the switch element W, the converter D of each pixel S is connected to a bias line LVS for supplying a reference voltage (bias voltage Vs here) to the pixel array 911. The bias line LVS is connected to a bias voltage supplier 918. Control of start and end of irradiation with radiation and the like may be performed using a current flowing the bias line LVS and detected by the bias voltage supplier 918.

In the pixel array 911, controls lines G1 to G3 corresponding to the first to third rows, and column signal lines LC1 to LC3 corresponding to the first to third columns are arranged. The driver 912 can drive the pixels S for each row using the control lines G1 to G3. For example, the control terminals (gate electrodes) of the switch elements W of the pixels S (1, 1), S (1, 2), and S (1, 3) are connected to the control line G1. When the driver 912 sets the control signal on the control line G1 at high level, the switch elements W of the pixels S (1, 1), S (1, 2), and S (1, 3) are turned on. With this, pixel signals corresponding to the electric charge amounts in the converters D are transferred from the pixels S (1, 1), S (1, 2), and S (1, 3) to the corresponding column signal lines LC1, LC2, and LC3, respectively.

In the following description, when the control lines G1 to G3 are not distinguished, they are simply referred to as the “control lines G”. This also applies to the other components.

The readout circuit 913 includes amplification units 1001 and a horizontal transfer unit 1002. Each amplification unit 1001 is arranged corresponding to each of the plurality of column signal lines LC, and includes an amplification circuit 1011 including an integration amplifier A1 and a variable amplifier A2, a sampling circuit 203, and a buffer circuit A3. In FIG. 10, a specific arrangement example of the amplification unit 1001 is shown only for the amplification unit 1001 corresponding to the column signal line LC1 in the first column, but the remaining amplification units 1001 can have the similar arrangement.

As shown in FIG. 10, the integration amplifier A1 includes an operational amplifier, a feedback capacitor arranged in a path between the inverting input terminal (“−” terminal in FIG. 10) and the output terminal of the operational amplifier, and a reset switch arranged in parallel to the feedback capacitor. A reference voltage VREF is supplied to the non-inverting input terminal (“+” terminal in FIG. 10) of the operational amplifier. While the reset switch is set in an OFF state, the pixel signal (more specifically, a potential fluctuation in the column signal line LC) output from the pixel S is amplified by the integration amplifier A1. When the reset switch is turned on, the integration amplifier A1 is reset.

The pixel signal amplified by the integration amplifier A1 is further amplified by a predetermined gain by the variable amplifier A2, and then sampled by the sampling circuit 203. The sampling circuit 203 includes a sampling switch and a holding capacitor connected to the sampling switch. When the sampling switch is turned on, the holding capacitor is connected to the output node of the amplification circuit 1011, and the holding capacitor is set at a voltage corresponding to the amplified pixel signal (sample). When the sampling switch is turned off, the holding capacitor is made to hold the voltage (hold).

The amplified pixel signal sampled as described above is horizontally transferred by the horizontal transfer unit 1002 via the buffer circuit A3. The horizontal transfer unit 1002 can be formed from, for example, a multiplexer and a shift register. When the horizontal transfer unit 102 sequentially selects the target column, the amplified pixel signal read out for each column is sequentially, horizontally transferred to the output circuit 914.

The output circuit 914 includes a buffer circuit A4 and an AD converter. The output circuit 914 amplifies the amplified pixel signal transferred horizontally by the buffer circuit A4, A/D-converts it by the AD converter, and outputs thus obtained signal to the controller 109 as image data.

In the arrangement shown in FIG. 10, by changing the feedback capacitor of the integration amplifier A1 and the gain of the variable amplifier A2, the sensitivity of the signal to be output can be changed. That is, by changing the amplification factor of the amplification circuit 1011, the sampling circuit 203 can sample the pixel signal with sensitivities different from each other.

In this embodiment, first of all, after a reset operation of the pixel array 911 is performed, accumulation of electric charges (to be sometimes referred to as first accumulation of electric charges hereinafter) is performed in each pixel S of the pixel array 911. More specifically, the switch element W of the pixel S is set in an OFF state for a predetermined period. With this operation, in the pixel S, electric charges corresponding to the radiation irradiation amount are accumulated in the converter D. In the first accumulation of electric charges, the feedback capacitor of the integration amplifier A1 is decreased, the gain of the variable amplifier A2 is increased, or both of them are performed. With this, the pixel signal can be acquired as a high-sensitivity signal ROH sampled with high sensitivity. At this time, the sampling circuit 203 simultaneously samples the pixel signals as the high-sensitivity signals ROH from the pixels S, of the plurality of pixels S, arranged in the row direction.

Then, after a reset operation of the pixel array 911 is performed, accumulation of electric charges (to be sometimes referred to as second accumulation of electric charges hereinafter) is performed in each pixel S of the pixel array 911. In the second accumulation of electric charges, the feedback capacitor of the integration amplifier A1 is increased, the gain of the variable amplifier A2 is decreased, or both of them are performed to set the sensitivity at low sensitivity. With this, the pixel signal can be acquired as a low-sensitivity signal ROL sampled with low sensitivity. At this time, the sampling circuit 203 simultaneously samples the pixel signals as the low-sensitivity signals ROL from the pixels S, of the plurality of pixels S, arranged in the row direction. The same radiation irradiation condition is used in the first accumulation of electric charges and the second accumulation of the electric charges.

When reading out of the high-sensitivity signals ROH and reading out of the low-sensitivity signals ROL are finished, the image processing illustrated in FIG. 6 is performed in the processor 130 of the controller 109 to generate a pixel value Pa of each pixel S. Thus, also in this embodiment, as in the first embodiment described above, it is possible to generate an image with improved linearity in a low dose region.

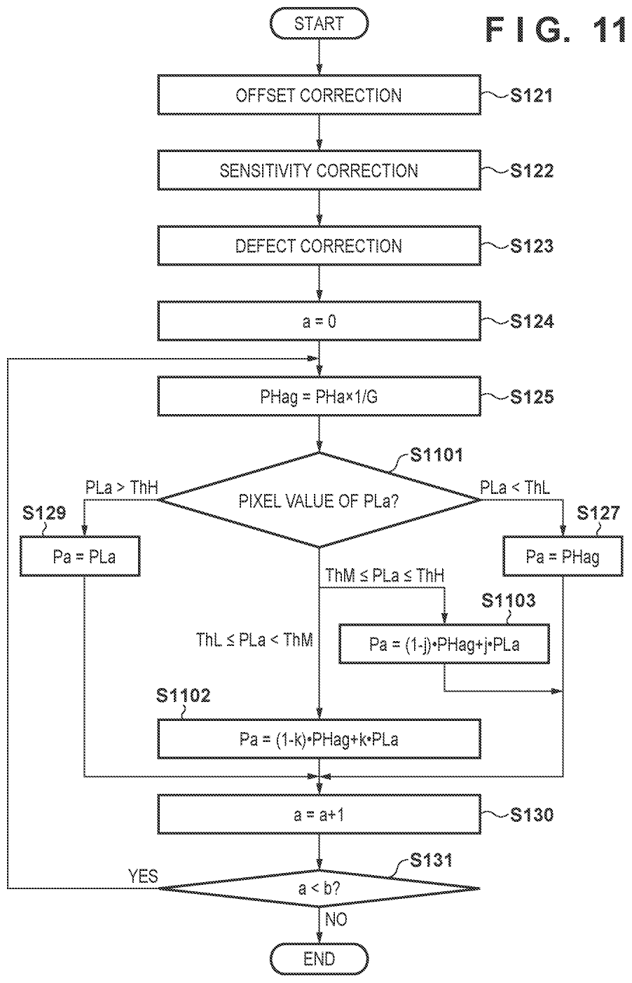

The example in which two threshold values ThL and ThH are used when generating the pixel value Pa has been described in each of the embodiments described above, but this is not exhaustive. For example, when generating a pixel value Pa, a processor 130 may select a method of generating the pixel value Pa using three or more threshold values. With reference to FIG. 11, processing will be described in which the processor 130 selects a method of generating the pixel value Pa using three threshold values.

Since steps S121 to S125 may be similar to the steps in FIG. 6 described above, a description thereof will be omitted here. In this embodiment, in order to select a method of generating the pixel value Pa, three threshold values are used in step S1101. Here, the three threshold values are a threshold value ThL, a threshold value ThH larger than the threshold value ThL, and an intermediate threshold value ThM between the threshold value ThL and the threshold value ThH (ThH>ThM>ThL).

In step S1101, if a low-sensitivity signal value PLa is smaller than the threshold value ThL, the process transitions to step S127. In step S127, as in the case described with reference to FIG. 6, the processor 130 sets, as the pixel value Pa of a pixel PIX at the pixel position “a”, a sensitivity adjustment value PHag which is obtained by adjusting, in accordance with a sensitivity rate G, a signal value PHa obtained by sampling with high sensitivity.

In step S1101, if the low-sensitivity pixel value PLa is larger than the threshold value ThH, the process transitions to step S129. In step S129, as in the case described with reference to FIG. 6, the processor 130 sets, as the pixel value Pa of the pixel PIX at the pixel position “a”, the signal value PLa obtained by sampling with low sensitivity.

In step S1101, if the low-sensitivity signal value PLa is equal to or larger than the threshold value ThL (equal to or larger than the first threshold value) and smaller than the threshold value ThM (smaller than the third threshold value), the process transitions to step S1102. In step S1102, the processor 130 generates the pixel value Pa of the pixel based on a weighted average value of the signal value PHa (sensitivity adjustment value PHag) obtained by sampling the optical signal (pixel signal) with high sensitivity by a sampling circuit 203 and the signal value PLa obtained by sampling with low sensitivity. More specifically, the processor 130 generates the pixel value Pa using a predetermined weighting coefficient k and following equation (3):

Pa=(1−k)×PHag+k×PLa (3)

The weighting coefficient k may be generated using, for example, following equation (4):

k=(PLa−ThL)/(ThM−ThL) (4)

In step S1101, if the low-sensitivity signal value PLa is equal to or larger than the threshold value ThM (equal to or larger than the third threshold value) and equal to or smaller than the threshold value ThH (equal to or smaller than the second threshold value), the process transitions to step S1103. In step S1103, the processor 130 generates the pixel value Pa of the pixel based on a weighted average value of the signal value PHa (sensitivity adjustment value PHag) obtained by sampling the optical signal (pixel signal) with high sensitivity by the sampling circuit 203 and the signal value PLa obtained by sampling with low sensitivity. More specifically, the processor 130 generates the pixel value Pa using a predetermined weighting coefficient j and following equation (5):

Pa=(1−j)×PHag+k×PLa (5)

The weighting coefficient j may be generated using, for example, following equation (6):

j=(PLa−ThM)/(ThH−ThM) (6)

In this manner, when acquiring the weighted average value of the signal value PHa and the signal value PLa, the weighting on the signal value PHa and the weighting on the signal value PLa may change with the threshold value ThM as the boundary. With this, it is possible to further smooth the boundary between an image based on the pixel value generated from the signal obtained by sampling with low sensitivity and an image based on the pixel value generated from the signal obtained by sampling with high sensitivity.

Since steps from step S130 may be similar to the steps in FIG. 6 described above, a description thereof will be omitted here. As in the case described above, the threshold values ThL, ThH, and ThM may be changeable in accordance with the radiation irradiation condition or the like. For example, the processor 130 may change the threshold values ThL, ThH, and ThM based on at least one of a dose to be applied during irradiation with radiation or an accumulation time, which are set by a user. Further, for example, the processor 130 may change the threshold values ThL, ThH, and ThM based on a signal value (to be sometimes referred to as a saturation value hereinafter) at which the optical signal (pixel signal) is saturated in sampling with high sensitivity, which is decided by the capacitance value of a capacitance Cfd or the like. For example, it may be set that threshold value ThL=(saturation value/G)×0.8, threshold value ThM=(saturation value/G)×0.9, and threshold value ThH=(saturation value/G)×1.0. Further, the relationship (linearity) between the incident radiation dose and the output signal value in sampling with low sensitivity may be measured in advance, and the threshold values ThL, ThH, and ThM may be decided based on the linearity.

As has been described above, when an imaging unit 100 of a radiation imaging apparatus 150 includes a memory 115, the memory 115 may store the threshold values ThL, ThH, and ThM corresponding to the radiation irradiation condition set by the user. In this case, the processor 130 may read out, from the memory 115, the threshold values ThL, ThH, and ThM in accordance with the irradiation condition, and use them to generate the pixel value Pa.

In the description of each of FIG. 6 and FIG. 11, the example has been described in which the pixel value Pa is generated assuming that the relationship (linearity) between the incident radiation dose and the output signal value is represented linearly. However, this is not exhaustive, and the pixel value Pa may be generated assuming that the relationship between the incident radiation dose and the output signal value is represented by a second- or higher-order function. This may be decided as needed in accordance with the characteristics of the linearity.

The present invention can be implemented by processing of supplying a program for implementing one or more functions of the above-described embodiments to a system or apparatus via a network or storage medium, and causing one or more processors in the computer of the system or apparatus to read out and execute the program. The present invention can also be implemented by a circuit (for example, an ASIC) for implementing one or more functions.

The means described above provides a technique advantageous in, in a radiation imaging apparatus, suppressing deterioration of the linearity in a low dose region in high dynamic range.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A radiation imaging apparatus that comprises a plurality of pixels each including a converter configured to generate a pixel signal corresponding to incident radiation, a sampling circuit configured to sample the pixel signal, and a processor configured to process a signal sampled by the sampling circuit, wherein
for each pixel of the plurality of pixels,
the sampling circuit is configured to sample the pixel signal with first sensitivity and with second sensitivity higher than the first sensitivity, and
if a first signal value obtained by sampling the pixel signal with the first sensitivity by the sampling circuit is smaller than a first threshold value, the processor is configured to generate a pixel value based on a second signal value obtained by sampling the pixel signal with the second sensitivity by the sampling circuit,
if the first signal value exceeds a second threshold value larger than the first threshold value, the processor is configured to generate a pixel value based on the first signal value, and
if the first signal value is not less than the first threshold value and not more than the second threshold value, the processor is configured to generate a pixel value based on the first signal value and the second signal value.

2. The radiation imaging apparatus according to claim 1, wherein
for each pixel of the plurality of pixels, if the first signal value is not less than the first threshold value and not more than the second threshold value, the processor is configured to generate a pixel value based on a weighted average value of the first signal value and the second signal value.

3. The radiation imaging apparatus according to claim 1, wherein
for each pixel of the plurality of pixels,
if the first signal value is not less than the first threshold value and smaller than a third threshold value between the first threshold value and the second threshold value, the processor is configured to generate a pixel value based on a first weighted average value of the first signal value and the second signal value,
if the first signal value is not less than the third threshold value and not more than the second threshold value, the processor is configured to generate a pixel value based on a second weighted average value of the first signal value and the second signal value, and
weighting on the first signal value and weighting on the second signal value change between a case in which the first weighted average value is acquired and a case in which the second weighted average value is acquired.

4. The radiation imaging apparatus according to claim 1, wherein
each of the plurality of pixels includes a switching capacitor, and a switch arranged between an output node of the converter and the switching capacitor,
when the switch is turned on, the sampling circuit is configured to sample the pixel signal with the first sensitivity, and
when the switch is turned off, the sampling circuit is configured to sample the pixel signal with the second sensitivity.

5. The radiation imaging apparatus according to claim 4, wherein
the sampling circuit includes a holding capacitor configured to hold a signal obtained by sampling the pixel signal with one of the first sensitivity and the second sensitivity, and
the holding capacitor is arranged in each of the plurality of pixels.

6. The radiation imaging apparatus according to claim 5, wherein
the holding capacitor includes a first holding capacitor configured to hold a signal obtained by sampling the pixel signal with the first sensitivity, and a second holding capacitor configured to hold a signal obtained by sampling the pixel signal with the second sensitivity.

7. The radiation imaging apparatus according to claim 5, wherein
the sampling circuit is configured to simultaneously sample the pixel signal with one of the first sensitivity and the second sensitivity in each of the plurality of pixels.

8. The radiation imaging apparatus according to claim 1, further including a plurality of column signal lines to which the pixel signals are transferred from the converters of the pixels, of the plurality of pixels, arranged in a column direction, and a readout circuit configured to read out the pixel signals transferred to the plurality of column signal lines,
wherein the readout circuit includes an amplification circuit corresponding to each of the plurality of column signal lines, and
by changing an amplification factor of the amplification circuit, the sampling circuit is configured to sample the pixel signal with one of the first sensitivity and the second sensitivity.

9. The radiation imaging apparatus according to claim 8, wherein
the amplification circuit comprises an integration amplifier including an operational amplifier and a feedback capacitor,
when the feedback capacitor is set at a first capacitance, the sampling circuit is configured to sample the pixel signal with the first sensitivity, and
when the feedback capacitor is set at a second capacitance larger than the first capacitance, the sampling circuit is configured to sample the pixel signal with the second sensitivity.

10. The radiation imaging apparatus according to claim 8, wherein
the amplification circuit includes a variable amplifier,
when a gain of the variable amplifier is set to a first gain, the sampling circuit is configured to sample the pixel signal with the first sensitivity, and
when the gain of the variable amplifier is set to a second gain smaller than the first gain, the sampling circuit is configured to sample the pixel signal with the second sensitivity.

11. The radiation imaging apparatus according to claim 8, wherein
the sampling circuit includes a holding capacitor configured to hold a signal obtained by sampling the pixel signal with one of the first sensitivity and the second sensitivity, and
the holding capacitor is connected to an output node of the amplification circuit.

12. The radiation imaging apparatus according to claim 8, wherein
the sampling circuit is configured to simultaneously sample the pixel signals with one of the first sensitivity and the second sensitivity from the pixels, of the plurality of pixels, arranged in a row direction.

13. The radiation imaging apparatus according to claim 1, wherein
the sampling circuit is capable of sampling the pixel signal with third sensitivity different from the first sensitivity and the second sensitivity.

14. The radiation imaging apparatus according to claim 1, wherein
the processor is configured to change the first threshold value and the second threshold value based on at least one of a dose to be applied during irradiation with radiation or an accumulation time, which are set by a user.

15. The radiation imaging apparatus according to claim 1, wherein
the processor is configured to change the first threshold value and the second threshold value based on a relationship, measured in advance, between an incident radiation dose to the converter and an output signal value in a case of the first sensitivity.

16. The radiation imaging apparatus according to claim 1, wherein
the processor is configured to decide the first threshold value and the second threshold value based on a signal value at which the pixel signal is saturated in a case of the second sensitivity.

17. A radiation imaging system comprising:
the radiation imaging apparatus according to claim 1; and
a radiation generation apparatus configured to apply radiation to the radiation imaging apparatus.

18. A drive method for a radiation imaging apparatus that comprises a plurality of pixels each including a converter configured to generate a pixel signal corresponding to incident radiation, the method including
sampling the pixel signal with first sensitivity and second sensitivity higher than the first sensitivity, and processing a signal sampled in the sampling the pixel,
wherein in the processing the signal, for each pixel of the plurality of pixels,
if a first signal value obtained by sampling the pixel signal with the first sensitivity is smaller than a first threshold value, a pixel value is generated based on a second signal value obtained by sampling the pixel signal with the second sensitivity,
if the first signal value exceeds a second threshold value larger than the first threshold value, a pixel value is generated based on the first signal value, and
if the first signal value is not less than the first threshold value and not more than the second threshold value, a pixel value is generated based on the first signal value and the second signal value.

19. A non-transitory computer-readable storage medium storing a program for causing a computer to execute a drive method for a radiation imaging apparatus that comprises a plurality of pixels each including a converter configured to generate a pixel signal corresponding to incident radiation, the method including
sampling the pixel signal with first sensitivity and second sensitivity higher than the first sensitivity, and processing a signal sampled in the sampling the pixel signal,
wherein in the processing the signal, for each pixel of the plurality of pixels,
if a first signal value obtained by sampling the pixel signal with the first sensitivity is smaller than a first threshold value, a pixel value is generated based on a second signal value obtained by sampling the pixel signal with the second sensitivity,
if the first signal value exceeds a second threshold value larger than the first threshold value, a pixel value is generated based on the first signal value, and
if the first signal value is not less than the first threshold value and not more than the second threshold value, a pixel value is generated based on the first signal value and the second signal value.
